(19) United States
(12) Patent Application Publication
(10) Pub. No.: US 2026/0256956 A1
(43) Pub. Date: Sep. 3, 2026

(54) STABILIZED RADIOPHARMACEUTICAL COMPOSITIONS

(71) Applicant: Jubilant Draximage Inc., Kirkland (CA)

(72) Inventors: Dinesh Sarwal Kumar, Greater Noida (IN); Arundhati Bhattacharyya, Greater Noida (IN); Vijayaraj Kuniyil Kulangara, Kirkland (CA); Indranil Nandi, Yardley, PA (US); Umamaheshwar Prasad, Kirkland (CA); Giuseppe Carlucci, Kirkland (CA)

(21) Appl. No.: 19/553,020

(22) Filed: Feb. 27, 2026

Related U.S. Application Data
(60) Provisional application No. 63/765,504, filed on Feb. 28, 2025.

(51) Int. Cl. A61K 51/04; A61K 47/02; A61K 47/12; A61K 47/26; A61K 47/34

(52) U.S. Cl. CPC A61K 51/0459; A61K 47/02; A61K 47/12; A61K 47/26; A61K 47/34; A61K 2123/00

(57) ABSTRACT

The present invention relates to stabilized radiopharmaceutical compositions comprising a F-18 radiolabelled compound and at least one stabilizer against radiolytic degradation. The disclosed compositions are free from cyclodextrin or derivatives thereof and in some embodiments are also free from ascorbic acid and its salts. More specifically, the present invention provides compositions comprising Flurpiridaz F-18 for myocardial perfusion imaging, with desirable technical attributes of purity, radiochemical purity, physical and chemical stability and shelf-life, comprising at least one stabilizer against radiolytic degradation and optionally a co-solvent or solubilizing agent wherein the compositions are free from cyclodextrin or derivatives thereof.

### FIELD OF THE INVENTION

The present invention relates to stabilized compositions comprising Fluorine-18 containing radiopharmaceutical compounds. The stabilized compositions are useful for diagnostic imaging using Positron Emission Tomography (PET). Specifically, this application relates to a stabilized composition of Flurpiridaz F-18 for PET imaging of myocardial perfusion.

### BACKGROUND

Coronary artery disease (CAD) is currently the leading cause of morbidity and mortality in developed nations. In the diagnosis of CAD, single-photon emission computed tomography (SPECT) based myocardial perfusion imaging agents (MPIA) has become an important tool used by cardiologists in the evaluation of regional myocardial blood flow and viability under rest or stress conditions. The most widely used SPECT MPIA in clinical settings are T1-201 or Tc-99m complexes, which are characterized by rapid myocardial extraction and by cardiac uptake proportional to blood flow. Positron Emission Tomography (PET) imaging has emerged as an alternative approach to evaluating myocardial blood flow by use of positron-emitting radionuclides. Although SPECT constitutes the primary modality for assessing myocardial perfusion in hospital settings, PET provides several advantages over SPECT imaging. These include higher special resolution, accurate attenuation correction, and the ability to quantify myocardial perfusion in absolute terms (milliliters per gram per minute) due to the physics inherent in the decay of positron-emitting radionuclides. Currently, three tracers are used for the assessment of myocardial perfusion with cardiac PET: ⁸²RbCl, ¹³NH₃, and H₂¹⁵O. Radiolabeled ammonia and water (¹³NH₃ and H₂¹⁵O) are produced using a cyclotron via the ¹⁶O (p, R)^{13N} and ¹⁵N(p, n)¹⁵O reactions, respectively, whereas ⁸²RbCl is produced from a Sr-82/Rb-82 generator. Owing to the short half-lives of the isotopes production of ¹³NH₃, and H₂¹⁵O must be performed in close proximity to a camera facility, which requires significant investment. The development of extractable perfusion imaging agents containing positron emitters with a sufficient half-life such as F-18 would therefore be highly desirable.

Fluorine-18 (F-18) is a positron emitting radioisotope of fluorine with a half-life of about 109.7 minutes. Fluorine-18 is presently the most frequently used radioisotope in positron emission tomography (PET) radiopharmaceuticals in both clinical and preclinical research. Its favorable characteristics such as 97% β+decay, 109.7 min half-life, 635 keV positron energy, along with high specific activity and ease of large scale production, makes it an attractive radionuclide for diagnostic PET imaging applications. In the last decade, several novel F-18 radiopharmaceuticals such as F-18 Fluciclovine, F-18 Florbetapir, F-18 Florbetaben, F-18 Piflufolastat, F-18 Flutemetamol, F-18 Flortaucipir, F-18 Fluoroestradiol and F-18 Flurpiridaz have been approved by USFDA for diverse indications like brain imaging, prostate cancer imaging, breast cancer imaging and cardiac imaging.

Flurpiridaz F-18 is a PET imaging agent indicated for myocardial perfusion imaging (MPI) under rest or stress (pharmacologic or exercise) in adult patients with known or suspected coronary artery disease (CAD) to evaluate for myocardial ischemia and infarction and is marketed under the brand name Flyrcado. According to the USFDA approved label, Flyrcado comprises hydroxypropyl-β-cyclodextrin as a solubilizer and co-radiostabilizer, L-(+)-ascorbic acid as a radiostabilizer and about 7% anhydrous ethanol by volume. It comprises 190 MBq/mL to 2,050 MBq/mL (5 mCi/mL to 55 mCi/mL) of Flurpiridaz F 18 at end of synthesis as a clear, colorless to yellow solution in a shielded multiple-dose vial with up to 30 mL fill volume. The pH of the solution is between 5.5 to 8.

It is desirable that F-18 radiopharmaceutical compositions should be able to maintain their chemical and radiochemical purity above the desirable level for at least 8-10 hours after end of synthesis. The radiolytic decomposition of F-18 radiopharmaceuticals is thought to be caused by hydroxyl free radicals resulting from the reaction of high energy positrons with the aqueous medium. Scott et al., (Appl Radiat Isot. 2009 January; 67 (1); 88-94) investigated the use of ethanol, ascorbic acid and nitrones as stabilizers for stabilization of F-18 radiopharmaceutical formulation. U.S. Pat. No. 9,687,571 discloses a composition comprising Flurpiridaz F-18 stabilized with ascorbic acid in the concentration range of 25 mg/mL and 500 mg/mL. United States patent publication no. 20220409752 discloses cyclodextrin (CD) as a co-stabilizer in F-18 radiopharmaceutical composition in conjunction with ascorbic acid and ethanol. There are challenges with use of ascorbic acid as a radioprotectant as ascorbic acid is susceptible to light and is prone to degradation in aqueous solution. Although cyclodextrins (CD), particularly β-CD are considered to be biocompatible, native CDs however, may pose toxicity concerns, particularly when administered by parenteral routes. The β-CD derivatives is known to cause undesirable toxicity issues, as HP-β-CD is ototoxic in vivo, due to its ability to destroy outer hair cells of the cochlea by extracting cholesterol from their membranes (Ferreira et al., European Journal of Pharmaceutics and Biopharmaceutics, September 2022; 178; 35-52).

The object of the present invention is to provide stabilized radiopharmaceutical compositions comprising an F-18 radiolabeled compound that is free of cyclodextrin and its derivatives. Another object of the present invention is to provide stabilized radiopharmaceutical compositions comprising an F-18 radiolabeled compound that is free of ascorbic acid and/or salts thereof. Another object of the present invention is to reduce pain and hemolysis at the site of injection caused by high concentration of ethanol by providing compositions comprising substantially reduced amount of ethanol. Another object of the present invention is to provide stabilized radiopharmaceutical compositions comprising improved shelf life and stability.

### SUMMARY

The present invention relates to a stabilized radiopharmaceutical compositions comprising an F-18 radiolabeled compound. The radiopharmaceutical compositions possess desirable technical attributes of purity, radiochemical purity, physical and chemical stability and shelf-life. The present invention also provides process of preparation of the radiopharmaceutical compositions. Method of use of the radiopharmaceutical compositions in PET imaging is also provided.

In one aspect, the present invention provides a stabilized radiopharmaceutical composition, comprising:
a) Flurpiridaz F-18;
b) an effective amount of at least one stabilizer selected from the group consisting of ascorbic acid, sodium ascorbate, gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, 5-hydroxy L-tryptophan, 2-aminothiazoline hydrobromide, S-(2-Aminoethyl)thiouronium bromide hydrobromide, sodium thiosulfate, sodium sulfite, meglumine and its derivatives, a polyaminoacid polymer, dimercaptosuccinic acid, vanillin, adenine, dobesilic acid, thymine, uracil, sodium metabisulfite, and combinations thereof;
wherein, the pH of the composition is from 4 to 9; and
wherein, the composition is free of cyclodextrin and derivatives thereof.

In one aspect of the present invention, the F-18 labeled compound is selected from F-18 Florbetapir, F-18 Florbetaben, F-18-Flortaucipir, F-18-Flutemetamol, F-18-Flurpiridaz, F-18-Piflufolastat, F-18 Flotufolastat, F-18 Fluciclovine, F-18 Fluorodopa, F-18 Fluoroestradiol, F-18 Fluoroethyl-L-tyrosine and F-18 Fluorodeoxyglucose.

In one aspect, the present invention provides a stabilized radiopharmaceutical composition, comprising:
a) Flurpiridaz F-18;
b) effective amount of at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, meglumine and its derivatives, a polyaminoacid polymer and combinations thereof;
wherein, the pH of the composition is from 4 to 9;
wherein, the composition is free of cyclodextrin and derivatives thereof;
wherein, the composition is free of ascorbic acid or a salt thereof;
wherein the total stabilizer concentration in the composition ranges from about 5 mg/ml to 100 mg/ml;
wherein, the bacterial endotoxin content in the composition is not more than 10 EU/ml;
wherein, the content of total impurities is not more than 5% and any individual impurities content is not more than 1%;
wherein the radioactivity concentration of the composition is in the range of 100 MBq/mL to 5000 MBq/mL;
wherein the initial radiochemical purity of the composition is at least 95% immediately after radiolabeling; and
wherein the radiochemical purity of the composition is at least 90% at 10 hours after end of synthesis when stored at a temperature ranging from 2° C. to 30° C.

In yet another aspect, the present invention provides stabilized radiopharmaceutical composition, comprising:
a) a Fluorine-18 labelled compound;
b) an effective amount of at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, 5-hydroxy L-tryptophan, 2-aminothiazoline hydrobromide, S-(2-Aminoethyl)thiouronium bromide hydrobromide, meglumine and its derivatives, a polyaminoacid polymer, dimercaptosuccinic acid, vanillin, adenine, dobesilic acid, thymine, uracil, sodium thiosulfate, sodium sulfite, sodium metabisulfite and combinations thereof;
wherein, the pH of the composition is from 4 to 9;
wherein, the composition is free of cyclodextrin and derivatives thereof;
wherein, the composition is free of ascorbic acid or a salt thereof;
wherein the initial radiochemical purity of the composition is at least 95% immediately after radiolabeling and wherein the shelf-life of the composition is at least 8 hours after end of synthesis when stored at a temperature ranging from 2° C. to 30° C.

### BRIEF DESCRIPTION OF THE DRAWINGS

FIG. 1: Illustrates typical F-18 labeling process flow for F-18 radiolabeled radiopharmaceutical products.

FIG. 2: Illustrates typical F-18 labeling process flow for Flurpiridaz F-18.

FIG. 3: Illustrates an alternative F-18 labeling process flow for Flurpiridaz F-18.

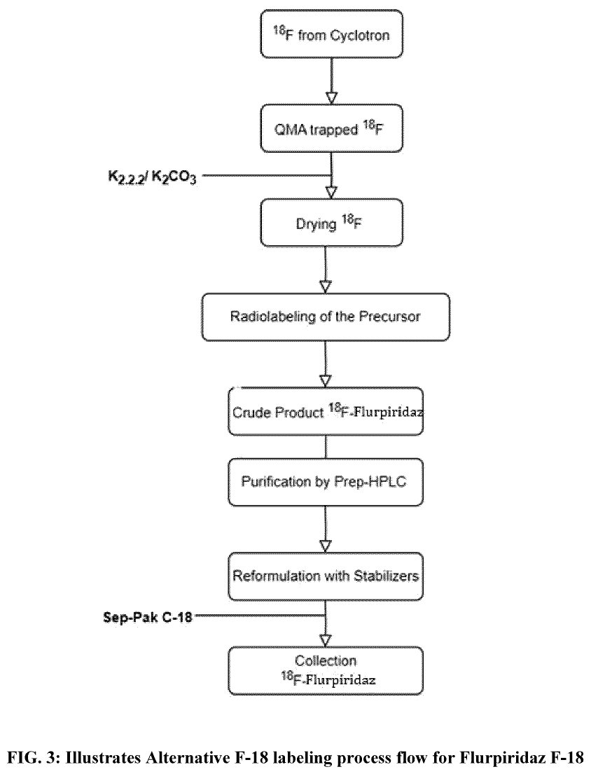

### DETAILED DESCRIPTION

The present invention can be more readily understood by reading the following detailed description of the invention and included embodiments.

The term “about” as used herein in the invention refers to a measurable value such as a parameter, an amount, a temporal duration, and the like, and is meant to encompass variations of and from the specified value, in particular variations of ±10% or less, preferably ±5% or less from the specified value, such variations are appropriate to perform in the disclosed invention. It is to be understood that the value to which the modifier “about” refers is itself also specifically, and preferably, disclosed.

As used in the specification of the present invention, the singular forms “a”, “an”, and “the” include plural references unless the context clearly dictates otherwise. Thus, for example, a reference to “a process” or “a composition” includes one or more process or composition, with one or more steps or ingredients or elements of the type described herein and/or which will become apparent to those persons skilled in the art upon reading this disclosure and so forth.

As used herein, the term “imaging” refers to techniques and processes used to create images of various parts of the human body for diagnostic and treatment purposes within digital health. Some imaging techniques are referred but are not limited to X-ray radiography, Fluoroscopy, Magnetic Resonance Imaging (MRI), Computed Tomography (CT), Medical Ultrasonography or Ultrasound Endoscopy, Elastography, Hybrid Molecular Imaging for Image Guided Surgery, Tactile Imaging, Thermography Medical Photography, and nuclear medicine functional imaging techniques, e.g. Positron Emission Tomography (PET), Dynamic Positron Emission Tomography or Single-Photon Emission Computed Tomography (SPECT). Imaging seeks to reveal internal structures of the body, as well as to diagnose and treat disease.

As used herein, the term “Positron Emission Tomography (PET)” refers to a functional imaging technique that uses radioactive substances known as radiotracers or radiopharmaceuticals to visualize and measure changes in metabolic processes, and in other physiological activities including blood flow, regional chemical composition, and absorption. Different radiotracers can be used for various imaging purposes, depending on the target process within the body. The radioisotopes typically used in PET imaging are carbon-11 (C-11), nitrogen-13 (N-13), oxygen-15 (O-15), fluorine-18 (F-18), rubidium-82 (Rb-82), copper-64 (Cu-64), zirconium-89 (Zr-89), and gallium-68 (Ga-68).

As used herein, the term “Cyclodextrins” refers to a family of cyclic oligosaccharides, consisting of a macrocyclic ring of glucose subunits joined by α-1,4 glycosidic bonds. Cyclodextrins are produced from starch by enzymatic conversion and are composed of 5 or more α-D-glucopyranoside units. Depending on the number of glucose subunits present, cylodextrins can be classified into three categories:
α (alpha)-cyclodextrin: consists of 6 glucose subunits
β (beta)-cyclodextrin: consists of 7 glucose subunits
γ (gamma)-cyclodextrin: consists of 8 glucose subunits

Cyclodextrins also include derivatives of natural cyclodextrins, such as hydroxypropyl-β-cyclodextrin (HBCD), methyl-β-cyclo-dextrin (MBCD) and sulfobutylether-β-cyclodextrin (SBEBCD).

As used herein, the term “dose” refers to the dose of a radionuclide required to perform imaging in a subject. The dose of a radionuclide to be administered into the subject ranges from 0.01 MBq to 10,000 MBq.

As used herein, the term “radionuclide” or “radioisotope” refers to an unstable form of a chemical element that releases radiation as it breaks down and becomes more stable. Radionuclides can occur in nature or can be generated in a laboratory. In medicine, they are used in imaging tests and/or in the treatment of diseases.

As used herein the term “radiochemical purity” refers to the proportion of the total radioactivity in the sample which is present as the desired radiolabelled species. Radiochemical purity is important since it is the radiochemical form, which determines the biodistribution of the radiopharmaceutical. The present invention includes the pharmaceutical composition, wherein the composition has a radiochemical purity of at least 80 percent, preferably 95 percent, most preferably 100 percent for 3 hours at room temperature. Radiochemical purity (RCP) is determined using radio TLC or HPLC and can be defined as the ratio of the (radio-labelled) drug substance peak to the total (radio-labelled) peaks in the chromatogram.

As used herein, the term “shelf-life” refers to the time period during which the quality parameters of the pharmaceutical product is expected to remain within the approved quality specifications for the product.

As used herein, the term “stabilizer” refers to a radio-stabilizer, which is a compound that inhibits radiolysis reactions by trapping highly-reactive free radicals, such as hydroxyl free radicals generated from the radiolysis of water. The stabilizers of the invention protect the radio-labelled compound(s) from radiolysis and therefore lower/prevent a drop in the purity of the radio-labelled compound(s) over their shelf life.

As used herein, the term “co-stabilizer” refers to a compound that enhances the desired effects of the stabilizer. Examples of co-stabilizer includes cyclodextrin and its derivatives.

As used herein, the term “impurities” refers to undesirable compounds formed in the radiopharmaceutical composition caused by free radical mediated radiolysis. Different type of impurities can form in F-18 radiolabeled compounds. Examples of such impurities include impurity B disclosed in PCT publication no. 2022263594.

As used herein, the term “subject” refers to a human or non-human mammal or animal. Non-human mammals include livestock animals, companion animals, laboratory animals, and non-human primates. Non-human subjects also specifically include, without limitation, horses, cows, pigs, goats, dogs, cats, mice, rats, guinea pigs, gerbils, hamsters, mink, and rabbits. In some embodiments of the invention, a subject is referred to as a “patient.” In some embodiments, a patient or subject may be under the care of a physician or other health care worker, including, but not limited to, someone who has consulted with, received advice from or received a prescription or other recommendation from a physician or other health care worker.

According to one embodiment, the present invention provides stabilized radiopharmaceutical composition, comprising:
a) a Fluorine-18 labelled compound;
b) an effective amount of at least one stabilizer selected from the group consisting of ascorbic acid, sodium ascorbate, gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, 5-hydroxy L-tryptophan, 2-aminothiazoline hydrobromide, S-(2-Aminoethyl)thiouronium bromide hydrobromide, meglumine and its derivatives, a polyaminoacid polymer, Dimercaptosuccinic acid, vanillin, adenine, dobesilic acid, thymine, uracil, sodium thiosulfate, sodium sulfite, sodium metabisulfite and combinations thereof;
wherein, the pH of the composition is from 4 to 9; and
wherein, the composition is free of cyclodextrin and derivatives thereof.

In one embodiment, the F-18 labeled compound is selected from F-18 Florbetapir, F-18 Florbetaben, F-18-Flortaucipir, F-18-Flutemetamol, F-18-Flurpiridaz, F-18-Piflufolastat, F-18 Flotufolastat, F-18 Fluciclovine, F-18 Fluorodopa, F-18 Fluoroestradiol, F-18 Fluoroethyl-L-tyrosine and F-18 Fluorodeoxyglucose.

In a preferred embodiment, the Fluorine-18 labeled compound is F-18 Flurpiridaz.

In another embodiment, the present invention provides stabilized radiopharmaceutical composition, comprising a Fluorine-18 labelled compound wherein, the composition comprises not more than 5% by volume of ethanol.

In another embodiment, the present invention provides stabilized radiopharmaceutical composition, comprising a Fluorine-18 labelled compound wherein the total stabilizer concentration ranges from about 0.01 mg/ml to 200 mg/ml, preferably 0.1 mg/ml to 150 mg/ml, more preferably 0.5 mg/ml to 100 mg/ml, more preferably 0.5 mg/ml to 50 mg/ml, more preferably 0.5 mg/ml to 60 mg/ml.

In another embodiment, the present invention provides stabilized radiopharmaceutical composition, comprising:
a) Flurpiridaz F-18;
b) an effective amount of at least one stabilizer selected from the group consisting of ascorbic acid, sodium ascorbate, gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, 5-hydroxy L-tryptophan, 2-aminothiazoline hydrobromide, S-(2-Aminoethyl)thiouronium bromide hydrobromide, sodium thiosulfate, sodium sulfite, meglumine and its derivatives, a polyaminoacid polymer, dimercaptosuccinic acid, vanillin, adenine, dobesilic acid, thymine, uracil, sodium metabisulfite, and combinations thereof;
wherein, the pH of the composition is from 4 to 9; and
wherein, the composition is free of cyclodextrin and derivatives thereof.

In another embodiment, the present invention provides stabilized radiopharmaceutical composition comprising Flurpiridaz F-18 wherein the total stabilizer concentration ranges from about 0.1 mg/ml to 100 mg/ml, more preferably from about 0.5 mg/ml to 80 mg/ml.

In another embodiment, the present invention provides stabilized radiopharmaceutical composition comprising Flurpiridaz F-18 wherein the initial radiochemical purity of the composition is at least 95% immediately after radiolabeling.

In another embodiment, the present invention provides stabilized radiopharmaceutical composition comprising Flurpiridaz F-18 wherein the shelf-life of the composition is at least 8 hours after end of synthesis when stored at a temperature ranging from 2° C. to 30° C.

In another embodiment, the present invention provides stabilized radiopharmaceutical composition comprising Flurpiridaz F-18 wherein the radiochemical purity of the composition is at least 90% at 10 hours after end of synthesis when stored at a temperature ranging from 2° C. to 30° C.

In another embodiment, the present invention provides stabilized radiopharmaceutical composition comprising Flurpiridaz F-18 wherein the radioactivity concentration of the composition is in the range of 100 MBq/mL to 5000 MBq/mL.

In another embodiment, the present invention provides stabilized radiopharmaceutical composition comprising Flurpiridaz F-18 wherein the composition further comprises a co-solvent selected from polyethylene glycol, propylene glycol, dimethyl sulfoxide (DMSO), dimethyl acetamide (DMA) and sorbitol.

In another embodiment, the present invention provides stabilized radiopharmaceutical composition comprising Flurpiridaz F-18 wherein the composition further comprises a solubilizer selected from non-ionic surfactants such as polyoxyethylene sorbitan monooleate, sorbitan monooloeate, polysorbates, Polyoxyethylene castor oil derivatives, polyethylene glycol stearate, polyethylene glycol ethers, poly(oxyethylene) poly(oxypropylene) poly(oxyethylene) block copolymers.

In another embodiment, the present invention provides stabilized radiopharmaceutical composition comprising Flurpiridaz F-18 wherein the composition comprises not more than 5% by volume of ethanol.

In another embodiment, the present invention provides stabilized radiopharmaceutical composition comprising Flurpiridaz F-18 wherein the composition is provided in a container selected from the group consisting of a vial, an ampoule, a prefilled syringe, and a capsule.

In another embodiment, the present invention provides stabilized radiopharmaceutical composition comprising Flurpiridaz F-18 wherein the composition is packed in a shielded Type I glass vial of size 1 ml to 100 ml.

In another embodiment, the present invention provides stabilized radiopharmaceutical composition comprising Flurpiridaz F-18 wherein the vial is shielded multiple dose vial of size up to 30 ml.

In another embodiment, the present invention provides a method of myocardial perfusion imaging comprising administering the radiopharmaceutical composition of claim 1 to a subject and acquiring an image of the subject by Positron Emission Tomography (PET).

In another embodiment, the present invention provides a stabilized radiopharmaceutical composition, comprising:
a) Flurpiridaz F-18;
b) effective amount of at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, meglumine and its derivatives, a polyaminoacid polymer and combinations thereof;
wherein, the pH of the composition is from 4 to 9;
wherein, the composition is free of cyclodextrin and derivatives thereof;
wherein, the composition is free of ascorbic acid or a salt thereof;
wherein the total stabilizer concentration in the composition ranges from about 5 mg/ml to 100 mg/ml;
wherein, the bacterial endotoxin content in the composition is not more than 10 EU/ml;

Wherein, the content of total impurities is not more than 5% and any individual impurities content is not more than 1%;
wherein the radioactivity concentration of the composition is in the range of 100 MBq/mL to 5000 MBq/mL;
wherein the initial radiochemical purity of the composition is at least 95% immediately after radiolabeling; and
wherein the radiochemical purity of the composition is at least 90% at 10 hours after end of synthesis when stored at a temperature ranging from 2° C. to 30° C.

In another embodiment, the present invention provides a process for the preparation of the radiopharmacetical composition comprising Flurpiridaz F-18, the process comprising the steps:
a) radiolabeling a precursor compound of Flurpiridaz F-18 with Fluorine-18 obtained from a cyclotron;
b) optionally purifying the reaction mixture by Solid Phase Extraction (SPE) method;
c) purifying the reaction mixture to remove impurities;
d) diluting the purified reaction mixture with aqeous buffered solution comprising at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, meglumine and its derivatives, a polyaminoacid polymer and combinations thereof; and
e) purification of the diluted bulk solution by Solid Phase Extraction (SPE) method; and
f) sterilizing the bulk solution by sterile filtration and dispensing into final product vials;
wherein, the process does not comprise addition of a cyclodextrin or derivative thereof; and
wherein, the process does not comprise addition of ascorbic acid or a salt thereof.

In another embodiment, the present invention provides kit for the preparation of a radiopharmaceutical composition comprising flurpiridaz F-18, comprising:
(i) a precursor compound for the production of Flurpiridaz F-18;
(ii) at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, meglumine and its derivatives, a polyaminoacid polymer and combinations thereof;
wherein, the kit does not comprise a cyclodextrin or derivative thereof; and
wherein, the kit does not comprise ascorbic acid or a salt thereof.

In another embodiment, the present invention provides physically stable radiopharmaceutical compositions comprising Fluorine-18 that has good compatibility with plastic contact materials including tubing, sterilizing filters, syringes etc. F-18 radiopharmaceutical compositions are prone to adsorption onto plastic contact materials which can lead to loss of activity during manufacturing. The present invention provides radiopharmaceutical compositions that show no obvious adsorption or loss of activity when tested by static and dynamic compatibility testing with plastic contact materials including tubing, sterilizing filters, syringes.

In another embodiment, the present invention provides stabilized radiopharmaceutical composition, comprising a Fluorine-18 labelled compound wherein the radioactivity concentration of the composition is in the range of 10 MBq/mL to 10,000 MBq/mL, preferably 100 MBq/mL to 5000 MBq/mL.

In another embodiment, the present invention provides stabilized radiopharmaceutical composition free of co-stabilizers such as cyclodextrin and derivatives thereof, comprising a Fluorine-18 labelled compound wherein the composition further comprises a poly-amino acid based amphiphilic polymer such as Apisolex™, which is a non-toxic, non-immunogenic, biocompatible, and biodegradable solubilizer.

In another embodiment, the present invention provides stabilized radiopharmaceutical composition, comprising a Fluorine-18 labelled compound wherein the composition is provided in a container selected from the group consisting of a vial, an ampoule, a prefilled syringe, and a capsule.

In another embodiment, the present invention provides stabilized radiopharmaceutical composition, comprising a Fluorine-18 labelled compound wherein the composition is packed in a Type I glass vial of size 1 ml to 100 ml.

In another embodiment, the present invention provides stabilized radiopharmaceutical composition, comprising a Fluorine-18 labelled compound wherein the composition is packed is a single dose vial or a multi dose vial.

In another embodiment, the present invention provides a method of positron emission tomography (PET) imaging comprising administering the stabilized radiopharmaceutical composition, comprising a Fluorine-18 labelled compound wherein the pH of the composition is within the range of about 4 to about 9 and wherein, the composition is free of cyclodextrin and derivatives thereof.

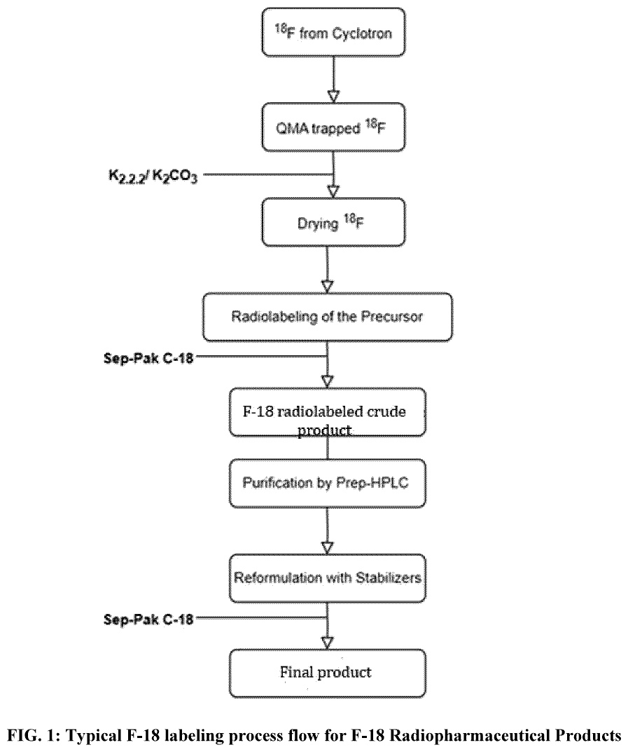

The radiosynthesis and formulations of Fluorine-18 radiolabeled compounds can be performed by the process depicted in FIG. 1.

The radiosynthesis of F-18 labeled Flurpiridaz can be performed by methods well known in prior art wherein cyclotron-produced F18-fluoride is trapped, dried, and chemically activated within a pre-assembled, disposable cassette that contains all required reagents, cartridges, and fluid pathways. The activated F18-fluoride then reacts with a precursor inside the cassette's reaction vessel to form the desired radiolabeled compound, after which integrated purification steps by solid-phase extraction (SPE) or semi-preparative HPLC to provide a formulation suitable for clinical or research use. The tosylate precursor compound for Flurpiridaz F-18 can be synthesized by methods disclosed in prior arts such as Purohit et. al. Journal of Medicinal Chemistry, 51 (10), 2954-2970) and Ahmed et. al., ChemMedChem. 2020 Jun. 17; 15 (12): 1040-1043.

### Example 1: Radiosynthesis of Flurpiridaz F-18

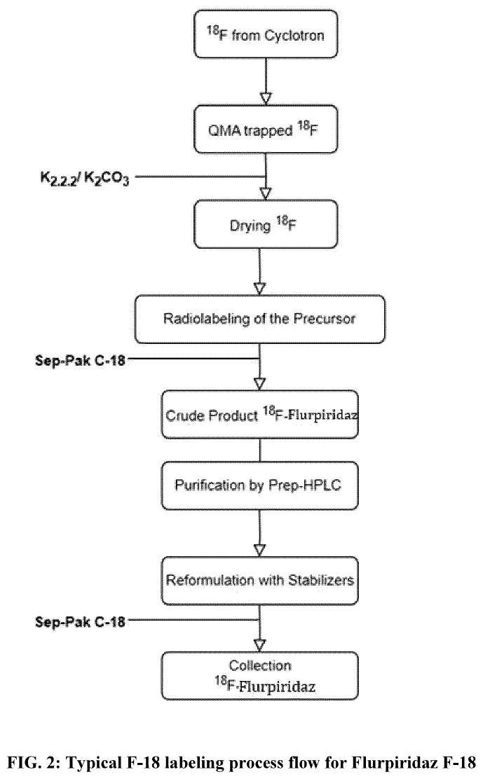

The radiosynthesis of Flurpiridaz F-18 is performed by nucleophilic radiofluorination method according to the method described by Purohit et. al. (Journal of Medicinal Chemistry, 51 (10), 2954-2970). First the tosylate precursor is reacted with kryptofix222/K18F complex and potassium carbonate in acetonitrile at 90° C. for 30 min followed by optional purification of the reaction mixture by Solid Phase Extraction (SPE) method. The reaction mixture is diluted with WFI and passed through Sep-Pak tC18 cartridge. After drying the cartridge with N2 gas, the cartridge is washed with water followed by 40% ethanol to remove the impurities. Then the SPE cartridge is eluted with a hydroalcoholic solvent which may additionally comprise a co-solvent and at least one stabilizer. After elution of the Flurpiridaz F-18, the product is diluted with buffered aqueous solution comprising at least one stabilizer. The bulk solution is then further purified by passing through the Sep-Pak Alumina N Plus Light Cartridge and two sterilization filters and dispensed into final product vials. The process of radiosynthesis is depicted in FIG. 1 and FIG. 2.

### Example 2: Purification of Flurpiridaz F-18

The method for purifying Flurpiridaz F-18 comprises subjecting the radiotracer to low- to medium-pressure liquid chromatography, wherein the chromatography is conducted using one or more than one disposable container filled with 2.55 g to 20 g of a stationary phase, wherein the stationary phase is selected from the group consisting of: silica, alumina A, alumina B, alumina N, magnesium silicate, magnesium oxide, zirconium oxide, C30, C18, tC18, C8, C4, C2, tC2, amino propyl, cyano propyl, diol, hydroxyapatite, cellulose, graphitized carbon, polystyrene/divinylbenzene polymers, polystyrene/divinylbezene copolymers and mixtures thereof, and wherein the low to medium-pressure liquid chromatography is conducted at a pressure of 1 to 20 bar.

### Example 3: Flurpiridaz F-18 Compositions Comprising Different Stabilizers

The quantitative compositions of the Flurpiridaz F-18 final formulations are shown in Table 1.

TABLE 1
FLURPIRIDAZ F-18 COMPOSITIONS COMPRISING DIFEERENT EXCIPIENTS

| | | Quantity (% w/v) | | | |
|---|---|---|---|---|---|
| | Example | Example | Example | Example | Example |
| Ingredient | 3 (a) | 3 (b) | 3 (c) | 3 (d) | 3 (e) |
| Flurpiridaz | 5 mCi-55 mCi | 5 mCi-55 mCi | 5 mCi-55 mCi | 5 mCi-55 mCi | 5 mCi-55 mCi |
| F-18 | ≤2.3 μg | ≤2.3 μg | ≤2.3 μg | ≤2.3 μg | ≤2.3 μg |
| | Flurpiridaz | Flurpiridaz | Flurpiridaz | Flurpiridaz | Flurpiridaz |
| Ascorbic acid | 0.1-7.5 | — | | — | — |
| Gentisic acid | — | 0.1-10 | 0.1-10 | — | — |
| Maleic acid | — | — | — | 0.5-10 | — |
| Benzyl alcohol | — | — | — | — | 0.1-2.5 |
| Meglumine | — | — | — | — | — |
| Methionine | — | — | — | — | — |
| Polyaminoacid | 0.01-10 | 0.01-10 | — | 0.01-10 | 0.01-10 |
| polymer (Apisolex) | | | | | |
| Sodium | 0.2-1 | 0.2-1 | 0.2-1 | 0.2-1 | 0.2-1 |
| hydroxide | | | | | |
| Anhydrous | 0.5-6 | 0.5-6 | 0.5-6 | 0.5-6 | 0.5-6 |
| Ethanol | | | | | |
| Water for | q.s. | q.s. | q.s. | q.s. | q.s. |
| Injection | | | | | |

| | Quantity (% w/v) | | | |
|---|---|---|---|---|
| | Example | Example | Example | Example |
| Ingredient | 3 (f) | 3 (g) | 3 (h) | 3 (i) |
| Flurpiridaz | 5 mCi-55 mCi | 5 mCi-55 mCi | 5 mCi-55 mCi | 5 mCi-55 mCi |
| F-18 | ≤2.3 μg | ≤2.3 μg | ≤2.3 μg | ≤2.3 μg |
| | Flurpiridaz | Flurpiridaz | Flurpiridaz | Flurpiridaz |
| Ascorbic acid | 0.1-7.5 | — | — | 0.1-7.5 |
| Gentisic acid | — | 0.1-10 | — | — |
| Maleic acid | — | — | — | — |
| Benzyl alcohol | — | — | — | — |
| Meglumine | 0.01-5 | 0.01-5 | — | — |
| Methionine | — | — | 0.1-5 | — |
| Polyaminoacid | — | — | 0.01-10 | — |
| polymer (Apisolex) | | | | |
| Sodium | 0.2-1 | 0.2-1 | 0.2-1 | 0.2-1 |
| hydroxide | | | | |
| Anhydrous | 0.5-6 | 0.5-6 | 0.5-6 | 0.5-6 |
| Ethanol | | | | |
| Water for | q.s. | q.s. | q.s. | q.s. |
| Injection | | | | |

General process of preparation of the compositions is outlined below:

The main Flurpiridaz F-18 product peak is collected from the preparative HPLC, it is diluted with a solution comprising the excipients selected from group comprising ascorbic acid, gentisic acid, meglumine, methionine, benzyl alcohol, Apisolex and/or combinations thereof, sodium hydroxide and then formulated in a 5-10% ethanol solution which may optionally comprise one or more stabilizer to obtain the final formulation.

The pH of the solution is from 4.5 to 8.5.

During routine preparation up to 1.65 Ci of the Flurpiridaz F-18 product is expected to be prepared.

### Example 4: Quality Control of Flurpiridaz F-18 Composition

The prepared compositions are subjected to various quality control tests as shown in Table 2 along with the method of testing and the desired quality attributes. The compositions of Example 3, more specifically the compositions of Example 3 (b), 3 (c) and 3 (g) are expected to conform to these quality characteristics.

TABLE 2
FLURPIRIDAZ F-18 QUALITY TARGET PRODUCT PROFILE

| Test | Method | Acceptance Criteria | |
|---|---|---|---|
| Appearance | Visual | Clear, Colorless, or light Yellow solution, | |
| | | free from visible contamination | |
| Bacterial | LAL test | NMT 10 EU/mL | |
| endotoxins | | | |
| Sterility | Sterility test | Sterile | |
| pH | pH indicator strip | 4.5-8.5 | |
| Ethanol content | Gas Chromatography | NMT 5% v/v | |
| | (GC) | | |
| Radiochemical | Radio HPLC | Flurpiridaz F-18 | NLT 96% |
| Purity | | Any other | NMT 1% |
| | | individual | |
| | | impurity | |
| | | Total impurities | NMT 5% |

| | | |
|---|---|---|
| Chemical Purity | HPLC | Flurpiridaz NLT 95% |
| by HPLC | | Any individual impurity NMT 1% |
| | | Total impurities NMT 5% |
| Radionuclidic | Gamma | ≥99.5% of total radioactivity as F-18 decay |
| Purity | Spectrometry | corrected to the time of expiration |
| Assay for | Gamma | 190 MBq/mL to 2,050 MBq/mL (5 mCi/mL to 55 |
| Radioactivity | Spectrometry | mCi/mL) of Flurpiridaz F 18 at end of |
| | | synthesis up to 30 mL fill volume |

### Example 5: Stability Studies of Flurpiridaz F-18 Composition

Radiochemical purity (RCP) of the prepared formulations are determined by Radio-HPLC at 2 hour intervals from the end of the synthesis till 12 hours post synthesis in ambient conditions. The product is expected to maintain RCP above 96% during this time.

What is claimed:

1. A stabilized radiopharmaceutical composition, comprising:
a) Flurpiridaz F-18;
b) an effective amount of at least one stabilizer selected from the group consisting of ascorbic acid, sodium ascorbate, gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, 5-hydroxy L-tryptophan, 2-aminothiazoline hydrobromide, S-(2-Aminoethyl)thiouronium bromide hydrobromide, sodium thiosulfate, sodium sulfite, meglumine and its derivatives, a polyaminoacid polymer, dimercaptosuccinic acid, vanillin, adenine, dobesilic acid, thymine, uracil, sodium metabisulfite, and combinations thereof;
wherein the pH of the composition is from 4 to 9; and
the composition is free of cyclodextrin and derivatives thereof.

2. The stabilized radiopharmaceutical composition of claim 1, wherein the total stabilizer concentration ranges from about 0.1 mg/ml to 100 mg/ml.

3. The stabilized radiopharmaceutical composition of claim 1, wherein the initial radiochemical purity of the composition is at least 95% immediately after radiolabeling.

4. The stabilized radiopharmaceutical composition of claim 1, wherein the shelf-life of the composition is at least 8 hours after end of synthesis when stored at a temperature ranging from 2° C. to 30° C.

5. The stabilized radiopharmaceutical composition of claim 1, wherein the radiochemical purity of the composition is at least 90% at 10 hours after end of synthesis when stored at a temperature ranging from 2° C. to 30° C.

6. The stabilized radiopharmaceutical composition of claim 1, wherein the radioactivity concentration of the composition is in the range of 100 MBq/mL to 5000 MBq/mL.

7. The stabilized radiopharmaceutical composition of claim 1, wherein the composition further comprises a co-solvent selected from polyethylene glycol, propylene glycol, dimethyl sulfoxide (DMSO), dimethyl acetamide (DMA) and sorbitol.

8. The stabilized radiopharmaceutical composition of claim 1, wherein the composition further comprises a solubilizer selected from non-ionic surfactants such as polyoxyethylene sorbitan monooleate, sorbitan monooloeate, polysorbates, Polyoxyethylene castor oil derivatives, polyethylene glycol stearate, polyethylene glycol ethers, poly(oxyethylene) poly(oxypropylene) poly(oxyethylene) block copolymers.

9. The stabilized radiopharmaceutical composition of claim 1, wherein the composition comprises not more than 5% by volume of ethanol.

10. The stabilized radiopharmaceutical composition of claim 1, wherein the composition is provided in a container selected from the group consisting of a vial, an ampoule, a prefilled syringe, and a capsule.

11. The stabilized radiopharmaceutical composition of claim 1, wherein the composition is packed in a shielded Type I glass vial of size 1 ml to 100 ml.

12. The stabilized radiopharmaceutical composition of claim 1, wherein the vial is a shielded multiple dose vial of size up to 30 ml.

13. A method of myocardial perfusion imaging comprising administering the stabilized radiopharmaceutical composition of claim 1 to a subject and acquiring an image of the subject by Positron Emission Tomography (PET).

14. A stabilized radiopharmaceutical composition, comprising:
a) Flurpiridaz F-18;
b) effective amount of at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, meglumine and its derivatives, a polyaminoacid polymer and combinations thereof;
wherein, the pH of the composition is from 4 to 9;
wherein, the composition is free of cyclodextrin and derivatives thereof;
wherein, the composition is free of ascorbic acid or a salt thereof;
wherein the total stabilizer concentration in the composition ranges from about 5 mg/ml to 100 mg/ml;
wherein, the bacterial endotoxin content in the composition is not more than 10 EU/ml;
wherein, the content of total impurities is not more than 5% and any individual impurities content is not more than 1%;
wherein the radioactivity concentration of the composition is in the range of 100 MBq/mL to 5000 MBq/mL;
wherein the initial radiochemical purity of the composition is at least 95% immediately after radiolabeling; and
wherein the radiochemical purity of the composition is at least 90% at 10 hours after end of synthesis when stored at a temperature ranging from 2° C. to 30° C.

15. The radiopharmaceutical composition of claim 14, wherein the composition further comprises a co-solvent selected from polyethylene glycol, propylene glycol, dimethyl sulfoxide (DMSO), dimethyl acetamide (DMA) and sorbitol.

16. The radiopharmaceutical composition of claim 14, wherein the composition further comprises a solubilizer selected from non-ionic surfactants such as polyoxyethylene sorbitan monooleate, sorbitan monooloeate, polysorbates, Polyoxyethylene castor oil derivatives, polyethylene glycol stearate, polyethylene glycol ethers, poly(oxyethylene) poly(oxypropylene) poly(oxyethylene) block copolymers.

17. A process for the preparation of the stabilized radiopharmaceutical composition of claim 16, the process comprising the steps:
a) radiolabeling a precursor compound of Flurpiridaz F-18 with Fluorine-18;
b) optionally purifying the reaction mixture by Solid Phase Extraction (SPE) method;
c) purifying the reaction mixture to remove impurities;
d) diluting the purified reaction mixture with aqueous buffered solution comprising at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, meglumine and its derivatives, a polyaminoacid polymer and combinations thereof; and
e) purification of the diluted bulk solution by Solid Phase Extraction (SPE) method; and
f) sterilizing the bulk solution by sterile filtration and dispensing into final product vials;
wherein, the process does not comprise addition of a cyclodextrin or derivative thereof; and
wherein, the process does not comprise addition of ascorbic acid or a salt thereof.

18. A kit for the preparation of the radiopharmaceutical composition of claim 14, comprising:
(i) a precursor compound for the production of Flurpiridaz F-18;
(ii) at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, meglumine and its derivatives, a polyaminoacid polymer and combinations thereof;
wherein, the kit does not comprise a cyclodextrin or derivative thereof; and
wherein, the kit does not comprise ascorbic acid or a salt thereof.

19. A method of myocardial perfusion imaging comprising administering the radiopharmaceutical composition of claim 14 to a subject and acquiring an image of the subject by Positron Emission Tomography (PET).

20. A stabilized radiopharmaceutical composition, comprising:
a) a Fluorine-18 labelled compound;
b) an effective amount of at least one stabilizer selected from the group consisting of gentisic acid, sodium gentisate, ascorbyl alcohol, gentisyl alcohol, maleic acid, cysteine, cystamine hydrochloride, methionine, niacinamide, nicotinic acid, p-amino benzoic acid, benzyl alcohol, 5-hydroxy L-tryptophan, 2-aminothiazoline hydrobromide, S-(2-Aminoethyl)thiouronium bromide hydrobromide, meglumine and its derivatives, a polyaminoacid polymer, dimercaptosuccinic acid, vanillin, adenine, dobesilic acid, thymine, uracil, sodium thiosulfate, sodium sulfite, sodium metabisulfite and combinations thereof;
wherein, the pH of the composition is from 4 to 9;
wherein, the composition is free of cyclodextrin and derivatives thereof;
wherein, the composition is free of ascorbic acid or a salt thereof;
wherein the initial radiochemical purity of the composition is at least 95% immediately after radiolabeling and wherein the shelf-life of the composition is at least 8 hours after end of synthesis when stored at a temperature ranging from 2° C. to 30° C.

21. The radiopharmaceutical composition of claim 20, wherein the F-18 labeled compound is selected from F-18 Florbetapir, F-18 Florbetaben, F-18-Flortaucipir, F-18-Flutemetamol, F-18-Flurpiridaz, F-18-Piflufolastat, F-18 Flotufolastat, F-18 Fluciclovine, F-18 Fluorodopa, F-18 Fluoroestradiol, F-18 Fluoroethyl-L-tyrosine and F-18 Fluorodeoxyglucose.

22. A method of diagnosis comprising administering the radiopharmaceutical composition of claim 20 to a subject and acquiring Positron Emission Tomography (PET) image of the subject.
